(19) United States
(12) Patent Application Publication
(10) Pub. No.: US 2024/0207221 A1
(43) Pub. Date: Jun. 27, 2024

(54) TREATMENT OF HYPERAMMONEMIA

(71) Applicant: BIOCODEX, Gentilly (FR)

(72) Inventors: Marc VERLEYE, Remy (FR); Philippe GIRARD, Margny-lès-Compiègne (FR); Marie-Emmanuelle LE GUERN, Compiègne (FR)

(21) Appl. No.: 18/556,499

(22) PCT Filed: Apr. 20, 2022
(86) PCT No.: PCT/EP22/60445

§ 371 Date: Oct. 20, 2023

(30) Foreign Application Priority Data
Apr. 20, 2021 (FR) FR2104119

(51) Int. Cl. A61K 31/357; A61K 45/06; A61P 3/00; A61P 25/08

(52) U.S. Cl. CPC A61K 31/357; A61K 45/06; A61P 3/00; A61P 25/08

(57) ABSTRACT

The present invention relates to a compound of the following formula (I):

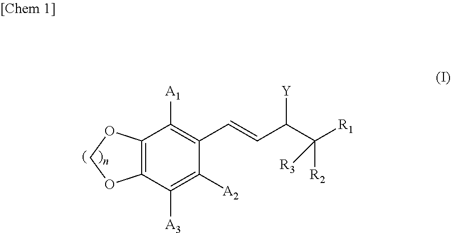

or a pharmaceutically acceptable salt, hydrate or solvate thereof, for use in the prevention or treatment of hyperammonemia in an individual.

### FIELD OF THE INVENTION

The present application relates to pharmaceutical compounds and compositions useful in the prevention or treatment of hyperammonemia, particularly drug-induced hyperammonemia.

### TECHNICAL BACKGROUND

Hyperammonemia is a metabolic disorder characterized by high levels of ammonia, a nitrogen-containing compound, in the blood. Ammonia is an important source of nitrogen and is required for amino acid synthesis. It is also necessary for normal acid-base balance. However, when present in high concentrations, ammonia is toxic.

Ammonia is normally produced in the colon and the small intestine, from where it is transported to the liver to be converted via the urea cycle into urea, a water-soluble compound which is then excreted by the kidneys.

The consequences of hyperammonemia are varied and can be serious. Ammonia is a potent neurotoxin, and its entry into the brain causes neurological disorders that can lead to convulsions, ataxia, stroke-like lesions, coma, psychosis, loss of vision, acute encephalopathy and cerebral edema. Other symptoms such as vomiting, respiratory alkalosis, hypothermia and liver damage leading to death are also observed in individuals with hyperammonemia.

Hyperammonemia must be detected early and treated immediately to avoid the development of potentially fatal complications such as cerebral edema or cerebral herniation.

Currently, the management of hyperammonemia involves a combination of measures to reduce ammonia levels in the blood. Typically, patients receive a low-protein diet combined with drugs intended to promote nitrogen elimination, such as sodium phenylbutyrate, sodium benzoate and glycerol phenylbutyrate (Enns et al., (2007) New Engl J Med., 356:2282-2292).

However, these drugs have to be administered several times a day and are associated with numerous side effects, in particular nausea, vomiting, irritability and anorexia. Moreover, because of the need to calculate dosages, overdosing can easily occur, leading to serious metabolic side effects and death.

Thus, there remains a need for effective alternative treatment and prevention of hyperammonemia.

### SUMMARY OF THE INVENTION

The present invention stems from the inventors' unexpected finding that stiripentol reduces methionine sulfoximine (MSO)-induced hyperammonemia, as well as methionine sulfoximine (MSO)-induced hyperammonemia-related seizures.

Thus, the present invention relates to a compound of the following formula (I):

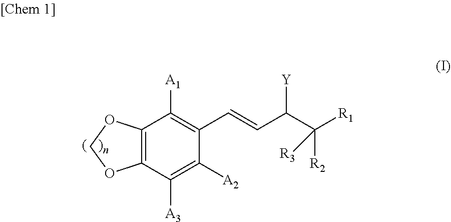

in which:
   n represents 1 or 2,
   A₁, A₂ and A₃, which may be identical or different, represent a hydrogen atom, a halogen atom, or a linear or branched alkyl group having 1 to 4 carbon atoms,
   R₁, R₂ and R₃ independently represent a hydrogen atom or a linear or branched alkyl group having 1 to 4 carbon atoms, and
   —Y represents —OH, ═O or —SH;
or a pharmaceutically acceptable salt, hydrate or solvate thereof, for use in the prevention or treatment of hyperammonemia in an individual.

The present invention also relates to the compound of formula (I) or the pharmaceutically acceptable salt hydrate or solvate thereof for use as defined above in combination with at least one additional compound for the prevention or treatment of hyperammonemia or for the prevention or treatment of one or more symptoms associated with hyperammonemia.

The present invention also relates to a pharmaceutical composition comprising as active substance at least one compound of formula (I) as defined above, or a pharmaceutically acceptable salt, hydrate or solvate thereof, optionally in association with a pharmaceutically acceptable carrier, for use in the prevention or treatment of hyperammonemia.

The present invention also relates to a pharmaceutical composition comprising as active substance at least one compound of formula (I) as defined above, or a pharmaceutically acceptable salt, hydrate or solvate thereof, optionally in association with a pharmaceutically acceptable carrier, and optionally comprising at least one additional compound for the prevention or treatment of hyperammonemia or for the prevention or treatment of one or more symptoms associated with hyperammonemia.

The present invention also relates to a pharmaceutical composition comprising as active substance at least one compound of formula (I) as defined above, or a pharmaceutically acceptable salt, hydrate or solvate thereof, optionally in association with a pharmaceutically acceptable carrier, and optionally comprising at least one additional compound for the prevention or treatment of hyperammonemia or for the prevention or treatment of one or more symptoms associated with hyperammonemia selected from the group consisting of neuroprotective drugs, anti-inflammatory drugs, sodium benzoate, sodium phenylbutyrate, glycerol phenylbutyrate, N-carbamylglutamate, sodium benzoate, sodium phenylacetate, lactulose, arginine hydrochloride, L-arginine, carglumic acid, L-ornithine, L-aspartate, citrulline and mixtures thereof.

The present invention also relates to products containing:
at least one compound of formula (I) as defined above, or a pharmaceutically acceptable salt, hydrate or solvate thereof, and
at least one additional compound for the prevention or treatment of hyperammonemia or for the prevention or treatment of one or more symptoms associated with hyperammonemia,
   as a combination product for use, in particular for simultaneous, separate or sequential use, in the prevention or treatment of hyperammonemia in an individual.

The present invention also relates to a method of preventing or treating hyperammonemia in an individual comprising administering to the individual an effective amount of a compound of formula (I) as defined above, or a pharmaceutically acceptable salt, hydrate or solvate thereof.

In an embodiment of the invention, the method as defined above also comprises the administration of at least one additional compound for the prevention or treatment of hyperammonemia or for the prevention or treatment of one or more symptoms associated with hyperammonemia.

The present invention also relates to the use of a compound of formula (I) as defined above, or a pharmaceutically acceptable salt, hydrate or solvate thereof, for the preparation of a medicament intended for the prevention or treatment of hyperammonemia in an individual.

In one embodiment of the invention, the medicament as defined above comprises at least one additional compound for the prevention or treatment of hyperammonemia or for the prevention or treatment of one or more symptoms associated with hyperammonemia.

### DETAILED DESCRIPTION OF THE INVENTION

As intended herein, the term “comprising” is synonymous with “including”, “containing” or “encompassing”, i.e. when an object “comprises” one or more features, features other than those mentioned may also be comprised in the object. Conversely, the expression “consisting of” means “constituted by”, i.e. when an object “consists of” one or more features, the object cannot include features other than those mentioned.

### Compound of Formula (I)

Preferably, the compound of formula (I) as defined above is represented by the following formula (II):

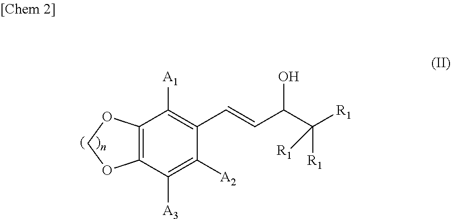

in which n, A₁, A₂, A₃ and R₁ are as defined above.

More preferably, the compound of formula (I) or (II) defined above is represented by the following formula (III):

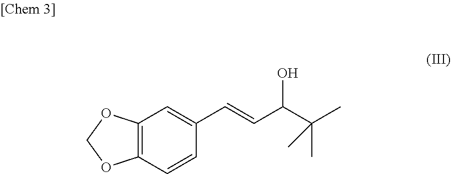 [SMILES: CC(C)(C)C(O)/C=C/c1ccc2c(c1)OCO2]

Preferred alkyl groups according to the invention include methyl, ethyl, n-propyl, isopropyl, n-butyl, s-butyl and t-butyl.

Cl, I, Br or F atoms are preferred halogen atoms according to the invention.

French patent FR 2 173 691, incorporated herein by reference, describes the synthesis of stiripentol, in particular from methylenedioxy-3,4-phenyl-1-dimethyl-4,4-penten-1-on-3. It is well within the ordinary skills of the skilled person to synthesize the other compounds of formula (I) from this teaching.

As will be clear to those skilled in the art, the formulae (I), (II) and (III) defined above represent either the various stereoisomers encompassed by these formulae, or mixtures thereof, in particular racemic mixtures thereof.

Thus, the compound of formula (III) can be a compound of formula (IIIa), a compound of formula (IIIb), or a mixture of a compound of formula (IIIa) and a compound of formula (IIIb), in particular the racemic mixture thereof.

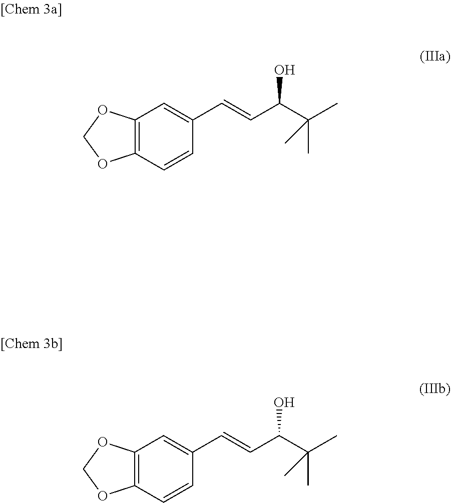 [SMILES: CC(C)(C)[C@H](O)/C=C/c1ccc2c(c1)OCO2 ; CC(C)(C)[C@@H](O)/C=C/c1ccc2c(c1)OCO2]

### Prevention or Treatment

“Hyperammonemia” refers to increased ammonia concentrations in the body. Thus, hyperammonemia according to the invention can be caused, for example, by an increase in ammonia production or by a decrease in ammonia detoxification processes in the body.

Ammonia (NH₃), which can coexist in the body with ammonium cation (NH₄⁺) depending on pH, is a product of the catabolism of proteins and other nitrogenated compounds. It is converted by the enzymes of the urea cycle into the less toxic substance urea, before being excreted in the urine by the kidneys.

The urea cycle, which also functions as a source for the production of certain amino acids (arginine, citrulline and ornithine) in the body, comprises 6 enzymes: carbamoyl phosphate synthase I (CPS), ornithine transcarbamylase (OTC), argininosuccinate synthetase (ASS), argininosuccinate lyase (ASL), arginase and N-acetylglutamate synthase (NAGS) and two transport proteins: ornithine translocase (ORNT1) and citrin. A defect can occur in one or more of these 8 proteins, resulting in hyperammonemia, also known as primary hyperammonemia.

Examples of urea cycle disorder leading to primary hyperammonemia according to the invention include:
N-acetylglutamate synthase deficiency;
carbamoylphosphate synthetase 1 deficiency;
ornithine transcarbamylase deficiency;
argininosuccinate synthetase deficiency;
argininosuccinate lyase deficiency;
arginase 1 deficiency;
hyperornithinemia-hyperammonemia-homocitrullinuria syndrome;
citrulline deficiency (type 2 citrullinemia).

Hyperammonemia according to the invention may also be due to inhibition of the urea cycle as a result of errors of intermediary metabolism characterized by reduced activity of enzymes or proteins that are not part of the urea cycle, also known as secondary hyperammonemia.

By way of example of an anomaly causing inhibition of the urea cycle leading to secondary hyperammonemia according to the invention, it is possible to cite:
propionic acidemia leading to glutamate deficiency;
methylmalonic acidemia leading to methylcitrate production;
isovaleric acidemia;
3-hydroxy-3-methylglutaryl-CoA-lyase deficiency;
ATP deficiency, leading to secondary CPS1 deficiency.
Another example of secondary hyperammonemia according to the invention is hyperammonemia caused by substrate deficiency, in particular by:
fatty acid oxidation defects leading to CoA deficiency;
disorders of the carnitine cycle leading to CoA deficiency;
disorders of the pyruvate dehydrogenase complex leading to CoA deficiency;
acute or chronic liver failure leading to CoA deficiency;
lysinuric protein intolerance leading to citrulline, arginine and ornithine deficiency;
hyperinsulinism-hyperammonemia syndrome leading to glutamate deficiency;
pyruvate carboxylase deficiency leading to aspartate deficiency;
pyrroline-5-carboxylate synthetase deficiency, leading to citrulline, arginine and ornithine deficiency.

Hyperammonemia according to the invention can also be linked to or caused by other pathologies, in particular pathologies selected from the group consisting of liver diseases, such as acute or chronic hepatic insufficiency, viral hepatitis, cirrhosis, impaired liver function, and vascular bypass of the liver resulting in reduced filtration of blood in the liver. This is also known as acquired hyperammonemia.

Hyperammonemia according to the invention can also result from the intake of medication or be due to chemotherapy, it is referred to as drug-induced hyperammonemia. Examples of drugs liable to cause hyperammonemia include sodium valproate, barbiturates such as primidone, glycine gel, L-asparaginase, 5-fluorouracil, sunitinib, asparaginase chemotherapy and regorafenib.

Hyperammonemia according to the invention can also result from the complication of a solid organ transplantation, such as a lung transplant, and is referred to as post-operative hyperammonemia.

Hyperammonemia prevented or treated according to the invention can also be the transient hyperammonemia of the newborn.

In an embodiment, the present invention relates to the prevention or treatment of disorders associated with hyperammonemia preferably selected from the group consisting of hyperammonemia-related convulsions and hyperammonemia-related convulsive seizures.

### Individual

The individual according to the invention is preferably a human being.

Preferably, the individual according to the invention suffers from hyperammonemia, in particular drug-induced hyperammonemia.

In one embodiment of the invention the individual is a full-term infant having a plasma ammonia concentration of at least 40 μM/L, at least 45 μM/L, at least 50 μM/L, at least 80 μM/L, or at least 90 μM/L.

In an embodiment of the invention, the individual is a premature infant with a plasma ammonia concentration of at least 70 μM/L, at least 80 μM/L or at least 90 μM/L.

In an embodiment of the invention the individual is a child over one month old having a plasma ammonia concentration of at least 40 μM/L, at least 45 μM/L, at least 50 M/L, at least 80 μM/L, or at least 90 μM/L.

In an embodiment of the invention, the individual is an adult with a plasma ammonia concentration of at least 30 μM/L, at least 40 μM/L at least 50 μM/L, at least 80 M/L, at least 150 μM/L, at least 180 μM/L, at least 200 μM/L, at least 300 μM/L or at least 400 μM/L.

In an embodiment of the invention, the individual according to the invention has a urea cycle disorder.

In an embodiment of the invention, the individual according to the invention presents a disturbance of cerebral functions. Preferably, the individual according to the invention presents at least one disorder selected from the group consisting of convulsions, convulsive seizures, confusion, agitation, disorders of consciousness, lethargy, ataxia, stroke-like lesions, coma, psychosis, loss of vision, acute encephalopathy, cerebral edema, and cerebral herniation. More preferably, the individual according to the invention has convulsions or convulsive seizures.

### Administration

Preferably, the compound of formula (I) as defined above, or its pharmaceutically acceptable salt, hydrate or solvate, is administered or administrable at a unit dose, or is packaged in a unit dose, of from about 50 mg to about 1500 mg, particularly of from about 150 mg to 300 mg. Also preferably, the compound of formula (I) as defined above or its pharmaceutically acceptable salt, hydrate or solvate is administered or administrable with a dosage regimen of from about 5 g/day to about 45 g/day. Preferably also the compound of formula (I) as defined above or its pharmaceutically acceptable salt, hydrate or solvate is administered or administrable with a dosage regimen of from 50 mg/kg/day to 500 mg/kg/day, preferably of from 250 mg/kg/d to 350 mg/kg/d, more preferably of about 300 mg/kg/d.

Preferably, the compound of formula (I) as defined above, or its pharmaceutically acceptable salt, hydrate or solvate, the pharmaceutical composition as defined above or the medicament as defined above is in a form acceptable for oral or rectal administration. Preferably, the compound of formula (I) as defined above, or its pharmaceutically acceptable salt, hydrate or solvate, the pharmaceutical composition as defined above or the medicament as defined above is administered or administrable in the form of a powder, tablets, capsules, sachets or suppositories.

In an embodiment, the compound of formula (I) as defined above, or its pharmaceutically acceptable salt, hydrate or solvate, the pharmaceutical composition as defined above or the medicament as defined above is not administered with another anticonvulsant or antiepileptic substance, such as sodium valproate and clobazam.

In an embodiment, the compound of formula (I) as defined above, or its pharmaceutically acceptable salt, hydrate or solvate, the pharmaceutical composition as defined above or the medicament as defined above is not administered with a carnitine treatment.

Preferably, the additional compound for the prevention or treatment of hyperammonemia or for the prevention or treatment of one or more symptoms associated with hyperammonemia according to the invention is intended to prevent or treat hyperammonemia, in particular by decreasing the production of ammonia in the blood or by increasing the mechanisms for eliminating ammonia from the blood.

Preferably also, the additional compound for the prevention or treatment of hyperammonemia or for the prevention or treatment of one or more symptoms associated with hyperammonemia can be any compounds intended to alleviate or treat one or more symptoms associated with hyperammonemia, such as neuroprotective or anti-inflammatory drugs.

Examples of symptoms associated with hyperammonemia include vomiting, loss of appetite, respiratory alkalosis, hypothermia, liver damage, neurological disorders such as convulsions, convulsive seizures, confusion, agitation, disturbed consciousness, lethargy, ataxia, stroke-like lesions, coma, psychosis, loss of vision, acute encephalopathy, cerebral edema, cerebral herniation.

Preferably, the additional compound useful for the prevention or treatment of hyperammonemia is selected from the group consisting of neuroprotective drugs such as N-acetylcysteine, indomethacin, propofol, minocycline and mannitol anti-inflammatory drugs, sodium benzoate, sodium phenylbutyrate, glycerol phenylbutyrate, N-carbamylglutamate, sodium benzoate/sodium phenylacetate, lactulose, arginine hydrochloride, L-arginine, carglumic acid, a mixture of L-ornithine and L-aspartate, citrulline, and mixtures thereof.

The compound of formula (I) as defined above, or its pharmaceutically acceptable salt, hydrate or solvate, the pharmaceutical composition as defined above or the medicament as defined above may also be associated with therapies intended to alleviate or reduce one or more symptoms associated with hyperammonemia or to prevent or treat hyperammonemia such as hemofiltration, intermittent hemodialysis, peritoneal dialysis, induced hypothermia, renal replacement therapy.

The compound of formula (I) as defined above, or its pharmaceutically acceptable salt, hydrate or solvate, the pharmaceutical composition as defined above or the medicament as defined above may also be combined with a low-protein diet and/or a diet in which caloric intakes are replaced by sugar and lipids.

As used herein, the expression “combined” or “in combination” or “combination product” means that the compound of formula (I) as defined above, or a pharmaceutically acceptable salt, hydrate or solvate thereof, in particular stiripentol, and the additional compound for the prevention or treatment of hyperammonemia or for the prevention or treatment of one or more symptoms associated with hyperammonemia as defined above, may be combined within the same pharmaceutical composition or medicament, and thus administered together, or be administered separately, i.e. by separate routes of administration and/or separate administration regimens, provided that when administered separately the period of time during which the compound of formula (I) as defined above or its pharmaceutically acceptable salt, hydrate or solvate exerts its pharmacological effects on the individual and the period of time during which the additional compound as defined above exerts its pharmacological effects on the individual overlap in whole or in part.

The invention will be further explained with the following non-limiting Examples and Figure.

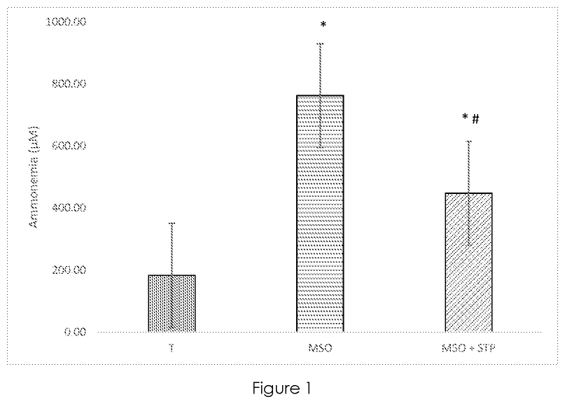

FIG. 1 shows the effect of intraperitoneally administered stiripentol (STP) on ammonia (expressed in μM, vertical axis) after treatment with 50 mg/kg methionine sulfoximine (MSO) in mice.

*p<0.05: ANOVA statistical test of treatment versus control groups.

#p<0.05: ANOVA statistical test of groups receiving MSO and stiripentol versus group receiving MSO only.

### Example

The inventors studied the efficacy of stiripentol on hyperammonemia and on hyperammonemia-associated convulsions in a model of methionine sulfoximine (MSO)-induced convulsions according to Hevor et al. (1985) Neuropathol. Appl. Neurobiol. 11: 129-139, as follows.

### A. Materials and Methods

### 1. Animals

Male CBA mice (Janvier) weighing between 23 and 27 grams are used after acclimatization for at least 4 days in the animal house (t°=22±2° C.; housing conditions are as follows: SAFE “A04” food; drinking water: tap water; nycthemeral cycle: 12 h/12 h (light: 7 h/19 h-dark: 19 h/7 h)).

On reception, mice are grouped in batches of 8 to 10 in 1500 U cages (floor area 1500 cm2) with bedding and sizzle dry (SDS) enrichment, in line with the ethical recommendations of European Parliament Directive 2010/63/EU (Sep. 22, 2010).

### 2. Protocol

On test day, mice are weighed, marked and placed in individual cages. At t ═O minutes, MSO (0.1 mL/10 g) is administered intraperitoneally, and the mice are carefully observed for 8 hours. Animals are normal for the first four hours. Tonic and clonic seizures appear for a few hours, then gradually disappear.

The parameters evaluated are the general condition of the animals, according to the Irwin grid (Irwin 1968), the number of convulsions, their onset times and mortality. Animals are euthanized as soon as limit points are reached, or at the end of the 8-hour observation period, according to the ANI07 procedure approved by the Ethics Committee.

Blood and whole brain with cerebellum samples will be taken in some studies to determine ammonia levels at sacrifice or at death for animals that do not complete the study.

Blood is collected under isoflurane anesthesia by cardiac puncture (0.5 to 1.0 mL per mouse) to determine ammonia levels. In the case of brain and blood sampling, the mouse is rapidly sacrificed by decapitation: the brain is removed and frozen in liquid nitrogen. The liver can then be removed and rapidly frozen in liquid nitrogen. Samples are stored at −80° C.

Stiripentol (0.1 mL/10 g) is administered intraperitoneally at different times with respect to MSO.

3. Ammonemia Measurement with the ABCAM Kit

Blood is collected after decapitation or by cardiac puncture, under isoflurane anesthesia (flow rate 1.5 l/min, evaporator at 4.3% for induction and maintenance) using an orange 25 G needle (Terumo, NN2516R) and a 1.3 ml S-Monovette tube containing EDTA (purple cap, ref 41.1395.105, Sarstedt). The tube is centrifuged at 2000 g for 10 minutes in a refrigerated 4° C. Beckman Allegra centrifuge. Plasma is collected in a 1 ml microtube (Brand, ref 780500). Microtubes are placed in a freezer at −80° C. before analysis, otherwise on crushed ice.

Ammonia is determined colorimetrically using a microplate reader (Dynex, model MRX), and an ABCAM kit (ref AB83360, ammonia assay kit) containing several reagents. This kit enables quantitative enzymatic determination of ammonium present in the samples. During the assay, ammonium is converted to a product that reacts with the OxiRed probe for quantification at 570 nm using a microplate reader. This test can detect 1 nmol (#20 μM) of ammonium. The kit, stored at −20° C., includes the following components:
Ammonia assay buffer (25 mL): ready to use, bring to room temperature before use;
OxiRed probe (200 μL): ready to use, warm to 37° ° C. to thaw DMSO;
Developer (1 vial): reconstitute with 220 μL of Ammonia assay buffer;
enzyme Mix (1 vial): reconstitute with 220 μL of Ammonia assay buffer;
Converting Enzyme (1 vial): reconstitute with 220 μl of Ammonia assay buffer,
10 mM NH₄Cl standard (100 μL): ready to use, keep on ice during use.
   Calibration Curve with the NH₄ Cl Standard
To be carried out every time you use it;
Prepare 100 μL of 1 mM NH₄Cl Standard;
10 μL of NH₄ Cl Standard at 10 mM+90 μL ED;
Using NH₄ Cl Standard at 1 mM, prepare dilutions according to the following Table 1:

TABLE 1

| | | | Final | | |
|---|---|---|---|---|---|
| | | | volume of | Final | |
| | Volume of | Assay | standard | ammonia | |
| | standard | buffer | in wells | concentration | |
| Standard | (μL) | (μL) | (μL) | in wells | |
| 1 | 0 | 150 | 50 | 0 | nmol/well |
| 2 | 6 | 144 | 50 | 2 | nmol/well |
| 3 | 12 | 138 | 50 | 4 | nmol/well |
| 4 | 18 | 132 | 50 | 6 | nmol/well |
| 5 | 24 | 126 | 50 | 8 | nmol/night |
| 6 | 30 | 120 | 50 | 10 | nmol/well |

### Table 1: Dilution Table

Standard at 1 mM, i.e. 1 mmol in 1 L or 1 nmol in 1 μL, so 6 nmol in 6 μl. Taking 50 μl out of 150 μL (6+144), we have 2 nmol/well.

### Sample Preparation:

Plasma or serum can be tested directly;
½, ⅕ and 1/10 dilutions;
A volume of 5-15 μl of plasma/serum per well.

### Ammonia Measurement Procedure:

Equilibrate all standards/samples/materials to room temperature;
Perform each measurement in duplicate;
Well filling;
   standard: 50 μL of standard dilution
   sample: 2 to 50 μl of sample and adjust to 50 μl with Assay Buffer
Prepare the reaction mix according to Table 2 below:

TABLE 2

| | Reaction mixture |
|---|---|
| Component | samples (μL) |
| Ammonia Assay Buffer | 42 |
| OxiRed Probe | 2 |
| Enzyme Mix | 2 |
| Developer | 2 |
| Converting Enzyme | 2 |

### Table 2: Preparation of the Reaction Mix

add 50 μL of reaction mix to standards and samples;
mix and incubate at 37° C. for 60 minutes, protected from light;
measure at 570 nm.

### Calculating Ammonia Concentrations

Values that are too high (outside the standard range) should be re-measured after dilution;
Averaging duplicate values;
Subtract the “blank” value to obtain the corrected OD;
Make a curve with the standards to obtain the equation, the slope;
Calculate the amount of ammonium in nmol/well (Sa)

Sa=(corrected OD−y)/slope
Calculate sample concentration (Sa/Sv)*D where Sv: sample volume; D: dilution factor; NH₄⁺ molar mass=18.04 g/mol.

### 4. Products

Methionine sulfoximine (Sigma, ref M5379, batches SLBN0115V, SLBP0971V) was solubilized in 0.9% NaCl.

Stiripentol (batch 176) was suspended in 5% (v/v) Tween 80 and 0.9% NaCl.

The ABCAM assay kit (ref AB83360, ammonia assay) and NH₄Cl (ammonium chloride, Sigma, ref 254134, molar mass 53.49 g) are used for ammonemia determinations.

### 5. Statistical Analysis

Results are expressed as the mean value±MSE. The statistical tests used are:
Fisher's exact test for percentages of convulsions and mortality, to determine a significant difference between groups at the 5% threshold,
A one-factor analysis of variance for the assays, to determine whether the pharmacological effect observed is significant at the 5% level (SigmaPlot 3.1 software).

### B. Results

### 1. Dosing Effect of Methionine Sulfoximine

Methionine sulfoximine (MSO) was administered intraperitoneally at different concentrations. Mouse behavior and mortality were observed up to 8 hours after administration:
At 40 mg/kg, MSO produced moderate behavioral effects: from the 4th hour onwards, hypotonia, hypomotility, accelerated respiration, ptosis and mydriasis were observed. At the end of observation (between 7 and 8 h), 2 mice out of 10 convulsed, and one died.
Starting at 50 mg/kg, signs of toxicity appear as early as the 3rd hour. Convulsions and mortality begin at 4 hours after MSO injection. 80% of mice convulsed, with a mortality rate of 90%.
At 60 mg/kg, methionine sulfoximine causes convulsions in 100% of mice, with a 90% mortality rate.
At 80 mg/kg, methionine sulfoximine causes convulsions in 100% of mice, with a 100% mortality rate.

Based on these results, a dose of 50 mg/kg methionine sulfoximine was chosen to study the protective effects of stiripentol on MSO-induced disorders.

### 2. Specific Effect of Stiripentol on Ammonemia

Stiripentol was administered intraperitoneally at 300 mg/kg without methionine sulfoximine.

It was observed that stiripentol did not alter ammonemia (176±14 μM) compared with the control group (179±11 μM).

3. Effects of Stiripentol after Administration of 50 mg/kg MSO

### 2.1. Stiripentol Administered Just Before MSO

Stiripentol is administered just before methionine sulfoximine according to Hevor et al. (1985) Neuropathol. Appl. Neurobiol. 11: 129-139.

Administration of 50 mg/kg methionine sulfoximine resulted in behavioral signs of toxicity, with a convulsion rate of 90% and a mortality rate of 75%.

Administration of 300 mg/kg stiripentol, just prior to MSO, significantly reduced the convulsion rate to 50% and the mortality rate to 25%, suggesting a protective effect.

In this study, ammonemia determinations gave the following results:
in controls not receiving methionine sulfoximine or any treatment, basal ammonemia is measured at 174±18 μM;
Administration of 50 mg/kg methionine sulfoximine resulted in a significant increase in ammonemia to 609±51 μM;
Administration of 300 mg/kg stiripentol, just prior to MSO, reduced this hyperammonemia by 32% to 417±51 μM.

### 2.2. Stiripentol Administered 30 Minutes Before MSO

Stiripentol is administered intraperitoneally 30 minutes before methionine sulfoximine according to Cloix et al. (2010). Epilepsia, 51: 118-128.

Administration of 50 mg/kg methionine sulfoximine resulted in behavioral signs of toxicity, with a convulsion rate of 100% and a mortality rate of 80%.

The administration of 300 mg/kg stiripentol, 30 minutes prior to MSO, significantly reduced the convulsion rate to 45% and the mortality rate to 25%, suggesting a protective effect.

During this study, ammonemia determinations gave the following results (FIG. 1):
In controls not receiving methionine sulfoximine or any treatment, ammonemia was measured at 184±12 μM,
Administration of 50 mg/kg methionine sulfoximine resulted in a significant increase in ammonemia to 763±71 μM,
Administration of 300 mg/kg stiripentol, 30 minutes prior to MSO, significantly reduced this hyperammonemia by 41% to 448±65 μM, compared with the MSO-only group.

### C. Conclusion

In the methionine sulfoximine (MSO)-induced convulsion model, the inventors have shown that a single intraperitoneal dose of 50 mg/kg MSO results in hyperammonemia, convulsions and mortality in 80-100% of mice.

The inventors have shown that intraperitoneal administration of stiripentol at 300 mg/kg significantly reduces methionine sulfoximine-induced convulsions and mortality, suggesting a protective effect.

MSO, which main mechanism of action is the irreversible inhibition of glutamine synthetase, an enzyme involved in ammonia detoxification, notably in the brain, leads to hyperammonemia (Ratnakumari, et al. (1985) J. Neurosci. Res. 14: 449-59). In astrocytes, glutamine synthetase uses ammonium and glutamate to manufacture glutamine, so inhibition of its activity by MSO leads to an increase in ammonia.

In terms of ammonemia, the inventors have shown that intraperitoneal administration of stiripentol at 300 mg/kg significantly reduces MSO-induced hyperammonemia.

1. A method for preventing or treating hyperammonemia in an individual, comprising administering a compound of the following formula (I): 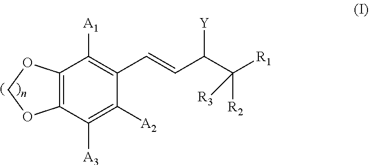
in which:
n represents 1 or 2,
A₁, A₂ and A₃, which may be identical or different, represent a hydrogen atom, a halogen atom, or a linear or branched alkyl group having 1 to 4 carbon atoms,
R₁, R₂ and R₃ independently represent a hydrogen atom or a linear or branched alkyl group having 1 to 4 carbon atoms, and
Y represents —OH, ═O or —SH;
or a pharmaceutically acceptable salt, hydrate or solvate thereof.

2. The method of claim 1, wherein the compound of formula (I) is of the following formula (II): 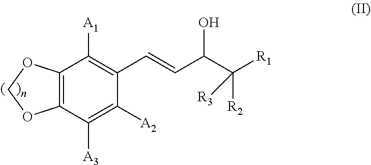
wherein n, A1, A2, A3 and R₁ are as defined in claim 1.

3. The method of claim 1, wherein the compound of formula (I) is of the following formula (III):

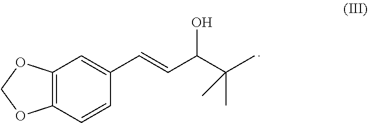 [SMILES: CC(C)(C)C(O)/C=C/c1ccc2c(c1)OCO2]

4. The method of claim 1, wherein said method is for the prevention or treatment of convulsions associated with hyperammonemia.

5. The method of claim 1, wherein said hyperammonemia is drug-induced hyperammonemia.

6. The method of claim 1, wherein said compound is packaged in a unit dose of from 50 mg to 1500 mg.

7. The method of claim 1, wherein said compound is suitable for oral administration.

8. The method of claim 1, wherein said compound is in the form of a powder, tablets, capsules, sachets or suppositories.

9. The method of claim 1, wherein said compound is administered in combination with at least one additional compound for the prevention or treatment of hyperammonemia or for the prevention or treatment of one or more symptoms associated with hyperammonemia.

10. The method of claim 1, wherein said compound is administered in combination with at least one additional compound for the prevention and treatment of hyperammonemia or for the prevention or treatment of one or more symptoms associated with hyperammonemia, selected from the group consisting of neuroprotective drugs, anti-inflammatory drugs, sodium benzoate, sodium phenylbutyrate, glycerol phenylbutyrate, N-carbamylglutamate, sodium benzoate, sodium phenylacetate, lactulose, arginine hydrochloride, L-arginine, carglumic acid, L-ornithine, L-aspartate, citrulline and mixtures thereof.

11. A pharmaceutical composition comprising as an active substance at least one compound of formula (I) of claim 1, or a salt, hydrate or solvate thereof, optionally in association with a pharmaceutically acceptable carrier, and optionally comprising at least one additional compound for the prevention or treatment of hyperammonemia or for the prevention or treatment of one or more symptoms associated with hyperammonemia.

12. A pharmaceutical composition comprising as an active substance at least one compound of formula (I) of claim 1, or a salt, hydrate or solvate thereof, optionally in association with a pharmaceutically acceptable carrier, and comprising at least one additional compound for the prevention or treatment of hyperammonemia or for the prevention or treatment of one or more symptoms associated with hyperammonemia selected from the group consisting of neuroprotective drugs, anti-inflammatory drugs, sodium benzoate, sodium phenylbutyrate, glycerol phenylbutyrate, N-carbamylglutamate, sodium benzoate, sodium phenylacetate, lactulose, arginine hydrochloride, L-arginine, carglumic acid, L-ornithine, L-aspartate, citrulline and mixtures thereof.

13. (canceled)
